(19) United States
(12) Patent Application Publication
(10) Pub. No.: US 2014/0058164 A1
(43) Pub. Date: Feb. 27, 2014

(54) Methane Conversion Apparatus and Process Using a Supersonic Flow Reactor

(71) Applicant: UOP LLC, Des Plaines, IL (US)

(72) Inventors: Gavin P. Towler, Inverness, IL (US); Laura E. Leonard, Western Springs, IL (US)

(73) Assignee: UOP LLC, Des Plaines, IL (US)

(21) Appl. No.: 13/964,425

(22) Filed: Aug. 12, 2013

Related U.S. Application Data
(60) Provisional application No. 61/691,341, filed on Aug. 21, 2012.

(51) Int. Cl. C07C 2/78

(52) U.S. Cl. CPC C07C 2/78
USPC 585/539

(57) ABSTRACT

Apparatus and methods are provided for converting methane in a feed stream to acetylene. A hydrocarbon stream is introduced into a supersonic reactor and pyrolyzed to convert at least a portion of the methane to acetylene. The reactor effluent stream may be treated to convert acetylene to another hydrocarbon process.

### CROSS-REFERENCE TO RELATED APPLICATION

This application claims priority from Provisional Application No. 61/691,341 filed Aug. 21, 2012, the contents of which are hereby incorporated by reference in its entirety.

### FIELD

Apparatus and methods are disclosed for converting methane in a hydrocarbon stream to acetylene using a supersonic flow reactor.

### BACKGROUND

Light olefin materials, including ethylene and propylene, represent a large portion of the worldwide demand in the petrochemical industry. Light olefins are used in the production of numerous chemical products via polymerization, oligomerization, alkylation and other well-known chemical reactions. These light olefins are essential building blocks for the modern petrochemical and chemical industries. Producing large quantities of light olefin material in an economical manner, therefore, is a focus in the petrochemical industry. The main source for these materials in present day refining is the steam cracking of petroleum feeds.

The cracking of hydrocarbons brought about by heating a feedstock material in a furnace has long been used to produce useful products, including for example, olefin products. For example, ethylene, which is among the more important products in the chemical industry, can be produced by the pyrolysis of feedstocks ranging from light paraffins, such as ethane and propane, to heavier fractions such as naphtha. Typically, the lighter feedstocks produce higher ethylene yields (50-55% for ethane compared to 25-30% for naphtha); however, the cost of the feedstock is more likely to determine which is used. Historically, naphtha cracking has provided the largest source of ethylene, followed by ethane and propane pyrolysis, cracking, or dehydrogenation. Due to the large demand for ethylene and other light olefinic materials, however, the cost of these traditional feeds has steadily increased.

Energy consumption is another cost factor impacting the pyrolytic production of chemical products from various feedstocks. Over the past several decades, there have been significant improvements in the efficiency of the pyrolysis process that have reduced the costs of production. In a typical or conventional pyrolysis plant, a feedstock passes through a plurality of heat exchanger tubes where it is heated externally to a pyrolysis temperature by the combustion products of fuel oil or natural gas and air. One of the more important steps taken to minimize production costs has been the reduction of the residence time for a feedstock in the heat exchanger tubes of a pyrolysis furnace. Reduction of the residence time increases the yield of the desired product while reducing the production of heavier by-products that tend to foul the pyrolysis tube walls. However, there is little room left to improve the residence times or overall energy consumption in traditional pyrolysis processes.

More recent attempts to decrease light olefin production costs include utilizing alternative processes and/or feed streams. In one approach, hydrocarbon oxygenates and more specifically methanol or dimethylether (DME) are used as an alternative feedstock for producing light olefin products. Oxygenates can be produced from available materials such as coal, natural gas, recycled plastics, various carbon waste streams from industry and various products and by-products from the agricultural industry. Making methanol and other oxygenates from these types of raw materials is well established and typically includes one or more generally known processes such as the manufacture of synthesis gas using a nickel or cobalt catalyst in a steam reforming step followed by a methanol synthesis step at relatively high pressure using a copper-based catalyst.

Once the oxygenates are formed, the process includes catalytically converting the oxygenates, such as methanol, into the desired light olefin products in an oxygenate to olefin (OTO) process. Techniques for converting oxygenates, such as methanol to light olefins (MTO), are described in U.S. Pat. No. 4,387,263, which discloses a process that utilizes a catalytic conversion zone containing a zeolitic type catalyst. U.S. Pat. No. 4,587,373 discloses using a zeolitic catalyst like ZSM-5 for purposes of making light olefins. U.S. Pat. Nos. 5,095,163; 5,126,308 and 5,191,141 on the other hand, disclose an MTO conversion technology utilizing a non-zeolitic molecular sieve catalytic material, such as a metal aluminophosphate (ELAPO) molecular sieve. OTO and MTO processes, while useful, utilize an indirect process for forming a desired hydrocarbon product by first converting a feed to an oxygenate and subsequently converting the oxygenate to the hydrocarbon product. This indirect route of production is often associated with energy and cost penalties, often reducing the advantage gained by using a less expensive feed material.

Recently, attempts have been made to use pyrolysis to convert natural gas to ethylene. U.S. Pat. No. 7,183,451 discloses heating natural gas to a temperature at which a fraction is converted to hydrogen and a hydrocarbon product such as acetylene or ethylene. The product stream is then quenched to stop further reaction and subsequently reacted in the presence of a catalyst to form liquids to be transported. The liquids ultimately produced include naphtha, gasoline, or diesel. While this method may be effective for converting a portion of natural gas to acetylene or ethylene, it is estimated that this approach will provide only about a 40% yield of acetylene from a methane feed stream. While it has been identified that higher temperatures in conjunction with short residence times can increase the yield, technical limitations prevent further improvement to this process in this regard.

While the foregoing traditional pyrolysis systems provide solutions for converting ethane and propane into other useful hydrocarbon products, they have proven either ineffective or uneconomical for converting methane into these other products, such as, for example ethylene. While MTO technology is promising, these processes can be expensive due to the indirect approach of forming the desired product. Due to continued increases in the price of feeds for traditional processes, such as ethane and naphtha, and the abundant supply and corresponding low cost of natural gas and other methane sources available, for example the more recent accessibility of shale gas, it is desirable to provide commercially feasible and cost effective ways to use methane as a feed for producing ethylene and other useful hydrocarbons.

### BRIEF DESCRIPTION OF THE DRAWINGS

FIG. 1 is a side cross-sectional view of a supersonic reactor in accordance with various embodiments described herein;

FIG. 2 is a schematic view of a system for converting methane into acetylene and other hydrocarbon products in accordance with various embodiments described herein; and

FIG. 3 is a side cross-sectional view of a supersonic reactor in accordance with various embodiments described herein.

### DETAILED DESCRIPTION

One proposed alternative to the previous methods of producing olefins that has not gained much commercial traction includes passing a hydrocarbon feedstock into a supersonic reactor and accelerating it to supersonic speed to provide kinetic energy that can be transformed into heat to enable an endothermic pyrolysis reaction to occur. Variations of this process are set out in U.S. Pat. Nos. 4,136,015 and 4,724,272, and Russian Patent No. SU 392723A. These processes include combusting a feedstock or carrier fluid in an oxygen-rich environment to increase the temperature of the feed and accelerate the feed to supersonic speeds. A shock wave is created within the reactor to initiate pyrolysis or cracking of the feed.

More recently, U.S. Pat. Nos. 5,219,530 and 5,300,216 have suggested a similar process that utilizes a shock wave reactor to provide kinetic energy for initiating pyrolysis of natural gas to produce acetylene. More particularly, this process includes passing steam through a heater section to become superheated and accelerated to a nearly supersonic speed. The heated fluid is conveyed to a nozzle which acts to expand the carrier fluid to a supersonic speed and lower temperature. An ethane feedstock is passed through a compressor and heater and injected by nozzles to mix with the supersonic carrier fluid to turbulently mix together at a speed of about Mach 2.8 and a temperature of about 427 C. The temperature in the mixing section remains low enough to restrict premature pyrolysis. The shockwave reactor includes a pyrolysis section with a gradually increasing cross-sectional area where a standing shock wave is formed by back pressure in the reactor due to flow restriction at the outlet. The shock wave rapidly decreases the speed of the fluid, correspondingly rapidly increasing the temperature of the mixture by converting the kinetic energy into heat. This immediately initiates pyrolysis of the ethane feedstock to convert it to other products. A quench heat exchanger then receives the pyrolized mixture to quench the pyrolysis reaction.

Methods and apparatus for converting hydrocarbon components in methane feed streams using a supersonic reactor are generally disclosed. As used herein, the term “methane feed stream” includes any feed stream comprising methane. The methane feed streams provided for processing in the supersonic reactor generally include methane and form at least a portion of a process stream. The apparatus and methods presented herein convert at least a portion of the methane to a desired product hydrocarbon compound to produce a product stream having a higher concentration of the product hydrocarbon compound relative to the feed stream.

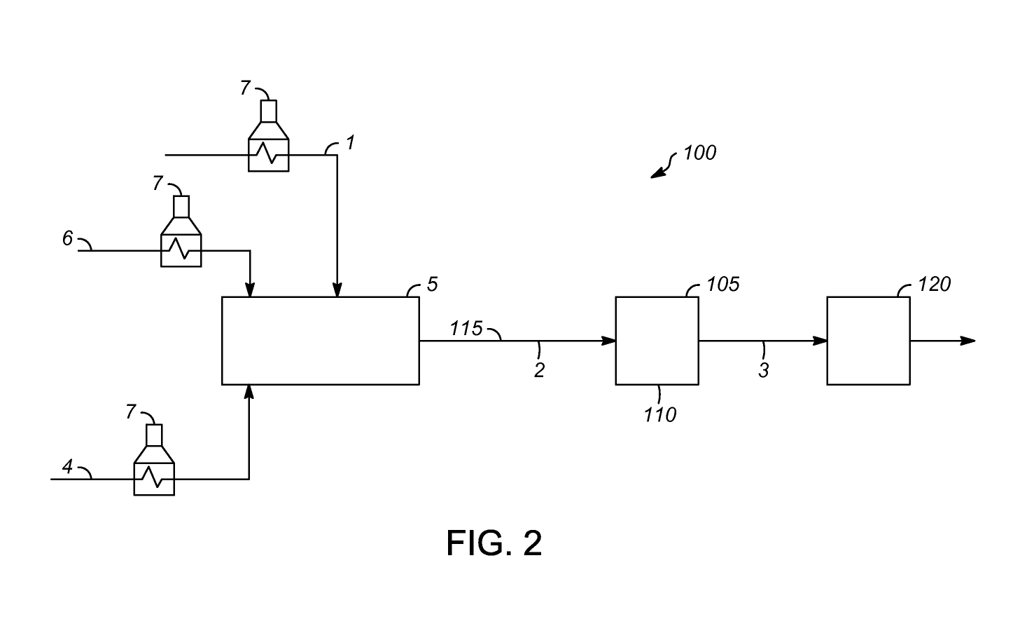

The term “hydrocarbon stream” as used herein refers to one or more streams that provide at least a portion of the methane feed stream entering the supersonic reactor as described herein or are produced from the supersonic reactor from the methane feed stream, regardless of whether further treatment or processing is conducted on such hydrocarbon stream. With reference to the example illustrated in FIG. 2, the “hydrocarbon stream” may include the methane feed stream 1, a supersonic reactor effluent stream 2, a desired product stream 3 exiting a downstream hydrocarbon conversion process or any intermediate or by-product streams formed during the processes described herein. The hydrocarbon stream may be carried via a process stream line 115, as shown in FIG. 2, which includes lines for carrying each of the portions of the process stream described above. The term “process stream” as used herein includes the “hydrocarbon stream” as described above, as well as it may include a carrier fluid stream, a fuel stream 4, an oxygen source stream 6, or any streams used in the systems and the processes described herein. The process stream may be carried via a process stream line 115, which includes lines for carrying each of the portions of the process stream described above. As illustrated in FIG. 2, any of methane feed stream 1, fuel stream 4, and oxygen source stream 6, may be preheated, for example, by one or more heaters 7.

Prior attempts to convert light paraffin or alkane feed streams, including ethane and propane feed streams, to other hydrocarbons using supersonic flow reactors have shown promise in providing higher yields of desired products from a particular feed stream than other more traditional pyrolysis systems. Specifically, the ability of these types of processes to provide very high reaction temperatures with very short associated residence times offers significant improvement over traditional pyrolysis processes. It has more recently been realized that these processes may also be able to convert methane to acetylene and other useful hydrocarbons, whereas more traditional pyrolysis processes were incapable or inefficient for such conversions.

The majority of previous work with supersonic reactor systems, however, has been theoretical or research based, and thus has not addressed problems associated with practicing the process on a commercial scale. In addition, many of these prior disclosures do not contemplate using supersonic reactors to effectuate pyrolysis of a methane feed stream, and tend to focus primarily on the pyrolysis of ethane and propane. One problem that has recently been identified with adopting the use of a supersonic flow reactor for light alkane pyrolysis, and more specifically the pyrolysis of methane feeds to form acetylene and other useful products therefrom, is the very large amount of heat that is produced in the supersonic reactor. In order to generate a large amount of heat and flowrate of the carrier fluid, a large amount of fuel is consumed. Further, at least a portion of the heat must be removed from the process stream after pyrolysis occurs in order to halt the reaction when the desired products have been produced in so that the reactor effluent and other streams may be sent downstream of the supersonic reactor. Moreover, additional heat may be required to preheat a fuel stream or a feed stream. Thus, it would be desirable, to reduce the amount of fuel and/or energy consumed by the supersonic reactor and to improve the overall efficiency thereof Previous work has not fully appreciated or addressed these concerns.

In addition, a carrier stream and feed stream may travel through the reactor at supersonic speeds, which can quickly erode many materials that could be used to form the reactor shell. Moreover, certain substances and contaminants that may be present in the hydrocarbon stream can cause corrosion, oxidation, and/or reduction of the reactor walls or shell and other equipment or components of the reactor. Such components causing corrosion, oxidation, or reduction problems may include, for example hydrogen sulfide, water, methanethiol, arsine, mercury vapor, carbidization via reaction with the fuel itself, or hydrogen embrittlement.

In accordance with various embodiments disclosed herein, therefore, apparatus and methods for converting methane in hydrocarbon streams to acetylene and other products are provided. Apparatus in accordance herewith, and the use thereof, have been identified to improve the overall process for the pyrolysis of light alkane feeds, including methane feeds, to acetylene and other useful products.

In accordance with one approach, the apparatus and methods disclosed herein are used to treat a hydrocarbon process stream to convert at least a portion of methane in the hydrocarbon process stream to acetylene. The hydrocarbon process stream described herein includes the methane feed stream provided to the system, which includes methane and may also include ethane or propane. The methane feed stream may also include combinations of methane, ethane, and propane at various concentrations and may also include other hydrocarbon compounds as well as contaminants. In one approach, the hydrocarbon feed stream includes natural gas. The natural gas may be provided from a variety of sources including, but not limited to, gas fields, oil fields, coal fields, fracking of shale fields, biomass, and landfill gas. In another approach, the methane feed stream can include a stream from another portion of a refinery or processing plant. For example, light alkanes, including methane, are often separated during processing of crude oil into various products and a methane feed stream may be provided from one of these sources. These streams may be provided from the same refinery or different refinery or from a refinery off gas. The methane feed stream may include a stream from combinations of different sources as well.

In accordance with the processes and systems described herein, a methane feed stream may be provided from a remote location or at the location or locations of the systems and methods described herein. For example, while the methane feed stream source may be located at the same refinery or processing plant where the processes and systems are carried out, such as from production from another on-site hydrocarbon conversion process or a local natural gas field, the methane feed stream may be provided from a remote source via pipelines or other transportation methods. For example a feed stream may be provided from a remote hydrocarbon processing plant or refinery or a remote natural gas field, and provided as a feed to the systems and processes described herein. Initial processing of a methane stream may occur at the remote source to remove certain contaminants from the methane feed stream. Where such initial processing occurs, it may be considered part of the systems and processes described herein, or it may occur upstream of the systems and processes described herein. Thus, the methane feed stream provided for the systems and processes described herein may have varying levels of contaminants depending on whether initial processing occurs upstream thereof.

In one example, the methane feed stream has a methane content ranging from about 65 mol-% to about 100 mol-%. In another example, the concentration of methane in the hydrocarbon feed ranges from about 80 mol-% to about 100 mol-% of the hydrocarbon feed. In yet another example, the concentration of methane ranges from about 90 mol-% to about 100 mol-% of the hydrocarbon feed.

In one example, the concentration of ethane in the methane feed ranges from about 0 mol-% to about 35 mol-% and in another example from about 0 mol-% to about 10 mol-%. In one example, the concentration of propane in the methane feed ranges from about 0 mol-% to about 5 mol-% and in another example from about 0 mol-% to about 1 mol-%.

The methane feed stream may also include heavy hydrocarbons, such as aromatics, paraffinic, olefinic, and naphthenic hydrocarbons. These heavy hydrocarbons if present will likely be present at concentrations of between about 0 mol-% and about 100 mol-%. In another example, they may be present at concentrations of between about 0 mol-% and 10 mol-% and may be present at between about 0 mol-% and 2 mol-%.

The apparatus and method for forming acetylene from the methane feed stream described herein utilizes a supersonic flow reactor for pyrolyzing methane in the feed stream to form acetylene. The supersonic flow reactor may include one or more reactors capable of creating a supersonic flow of a carrier fluid and the methane feed stream and expanding the carrier fluid to initiate the pyrolysis reaction. In one approach, the process may include a supersonic reactor as generally described in U.S. Pat. No. 4,724,272, which is incorporated herein by reference, in its entirety. In another approach, the process and system may include a supersonic reactor such as described as a “shock wave” reactor in U.S. Pat. Nos. 5,219,530 and 5,300,216, which are incorporated herein by reference, in their entirety. In yet another approach, the supersonic reactor described as a “shock wave” reactor may include a reactor such as described in “Supersonic Injection and Mixing in the Shock Wave Reactor” Robert G. Cerff, University of Washington Graduate School, 2010.

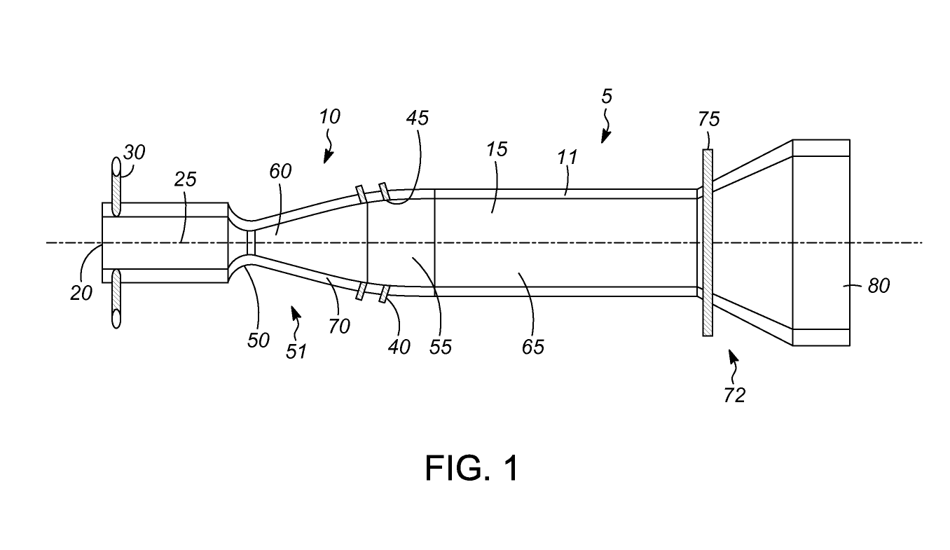

While a variety of supersonic reactors may be used in the present process, an exemplary reactor 5 is illustrated in FIG. 1. Referring to FIG. 1, the supersonic reactor 5 includes a reactor vessel 10 generally defining a reactor chamber 15. While the reactor 5 is illustrated as a single reactor, it should be understood that it may be formed modularly or as separate vessels. If formed modularly or as separate components, the modules or separate components of the reactor may be joined together permanently or temporarily, or may be separate from one another with fluids contained by other means, such as, for example, differential pressure adjustment between them. A combustion zone or chamber 25 is provided for combusting a fuel to produce a carrier fluid with the desired temperature and flowrate. The reactor 5 may optionally include a carrier fluid inlet 20 for introducing a supplemental carrier fluid into the reactor. One or more fuel injectors 30 are provided for injecting a combustible fuel, for example hydrogen, into the combustion chamber 25. The same or other injectors may be provided for injecting an oxygen source into the combustion chamber 25 to facilitate combustion of the fuel. The fuel and oxygen are combusted to produce a hot carrier fluid stream typically having a temperature of from about 1200 to about 3500 C in one example, between about 2000 and about 3500 in another example, and between about 2500 and about 3200 C in yet another example. It is also contemplated herein to produce the hot carrier fluid stream by other known methods, including non-combustion methods. According to one example the carrier fluid stream has a pressure of about 1 atm or higher, greater than about 2 atm in another example, and greater than about 4 atm in another example.

The hot carrier fluid stream from the combustion zone 25 is passed through a supersonic expander 51 that includes a converging-diverging nozzle 50 to accelerate the flowrate of the carrier fluid to above about mach 1.0 in one example, between about mach 1.0 and mach 4.0 in another example, and between about mach 1.5 and 3.5 in another example. In this regard, the residence time of the fluid in the reactor portion of the supersonic flow reactor is between about 0.5-100 ms in one example, about 1.0-50 ms in another example, and about 1.5-20 ms in another example. The temperature of the carrier fluid stream through the supersonic expander by one example is between about 1000 C and about 3500 C, between about 1200 C and about 2500 C in another example, and between about 1200 C and about 2000 C in another example.

A feedstock inlet 40 is provided for injecting the methane feed stream into the reactor 5 to mix with the carrier fluid. The feedstock inlet 40 may include one or more injectors 45 for injecting the feedstock into the nozzle 50, a mixing zone 55, a diffuser zone 60, or a reaction zone or chamber 65. The injector 45 may include a manifold, including for example a plurality of injection ports or nozzles for injecting the feed into the reactor 5.

In one approach, the reactor 5 may include a mixing zone 55 for mixing of the carrier fluid and the feed stream. In one approach, as illustrated in FIG. 1, the reactor 5 may have a separate mixing zone, between for example the supersonic expander 51 and the diffuser zone 60, while in another approach, the mixing zone is integrated into the diffuser section, and mixing may occur in the nozzle 50, expansion zone 60, or reaction zone 65 of the reactor 5. An expansion zone 60 includes a diverging wall 70 to produce a rapid reduction in the velocity of the gases flowing therethrough, to convert the kinetic energy of the flowing fluid to thermal energy to further heat the stream to cause pyrolysis of the methane in the feed, which may occur in the expansion section 60 and/or a downstream reaction section 65 of the reactor. The fluid is quickly quenched in a quench zone 72 to stop the pyrolysis reaction from further conversion of the desired acetylene product to other compounds. Spray bars 75 may be used to introduce a quenching fluid, for example water or steam into the quench zone 72.

The reactor effluent exits the reactor via outlet 80 and as mentioned above forms a portion of the hydrocarbon stream. The effluent will include a larger concentration of acetylene than the feed stream and a reduced concentration of methane relative to the feed stream. The reactor effluent stream may also be referred to herein as an acetylene stream as it includes an increased concentration of acetylene. The acetylene stream may be an intermediate stream in a process to form another hydrocarbon product or it may be further processed and captured as an acetylene product stream. In one example, the reactor effluent stream has an acetylene concentration prior to the addition of quenching fluid ranging from about 2 mol-% to about 30 mol-%. In another example, the concentration of acetylene ranges from about 5 mol-% to about 25 mol-% and from about 8 mol-% to about 23 mol-% in another example.

The reactor vessel 10 includes a reactor shell 11. It should be noted that the term “reactor shell” refers to the wall or walls forming the reactor vessel, which defines the reactor chamber 15. The reactor shell 11 will typically be an annular structure defining a generally hollow central reactor chamber 15. The reactor shell 11 may include a single layer of material, a single composite structure or multiple shells with one or more shells positioned within one or more other shells. The reactor shell 11 also includes various zones, components, and or modules, as described above and further described below for the different zones, components, and or modules of the supersonic reactor 5. The reactor shell 11 may be formed as a single piece defining all of the various reactor zones and components or it may be modular, with different modules defining the different reactor zones and/or components.

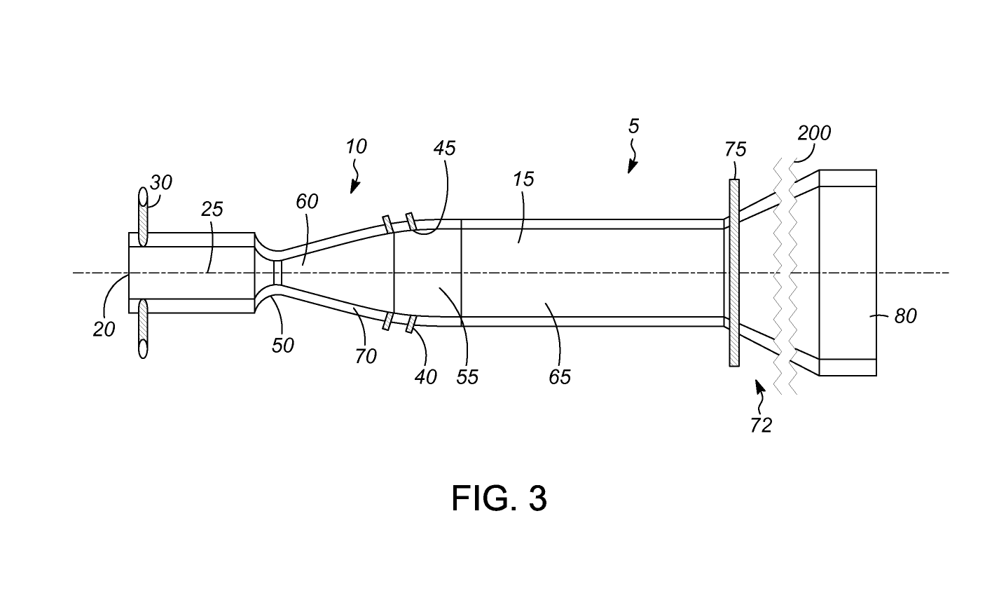

By one approach, as illustrated in FIG. 3, at least one heat exchanger 200 is provided for transferring heat from at least a portion of the supersonic reactor pyrolysis or effluent stream to one or more other portions of the process stream.

In one approach, the heat exchanger 200 may be generally downstream of the supersonic reactor 5 such that a reactor effluent line carrying at least a portion of the reactor effluent provides fluid to the heat exchanger 200. In another approach, the heat exchanger 200 may be integrated with the supersonic reactor 5, including adding a portion thereof within the reactor chamber. In this approach, the heat exchanger 200 may include a stab-in heat exchanger In this regard, the heat exchanger 200 or a portion thereof may be positioned within various locations of the supersonic reactor, including, for example, within the reaction zone 65 or the quench zone 70 to transfer heat from at least one of the pyrolysis stream and the effluent stream flowing through the reactor chamber 15. The stab-in heat exchanger includes a heat transfer fluid flowing therethrough. The heat transfer fluid may include various heat transfer fluids known in the art, including, but not limited to molten metal VSO heat exchanger fluid, raising stream, superheating steam, hot oil, and liquid sodium

By one approach, in order to withstand harsh operating conditions within the reactor chamber 15, at least a portion of the heat exchanger may include a ceramic tube heat exchanger. In one approach, the ceramic tube heat exchanger comprises a material selected from the group of a carbide, a nitride, titanium diboride, a sialon ceramic, zirconia, or thoria. In another approach, the heat exchanger 200 includes highly inert coated tubes to restrict corrosion of the tubes within the reactor chamber 15. In one approach, the highly-inert tubes include highly-sulfided tubes that are formed by sulfiding. In another approach, the highly-inert tubes include carbon-carbon tubes. A carbon-carbon tubes may be formed by providing a carbon coating on one or more tubes.

In one approach, the heat exchanger 200 includes tubes formed of a material having a melting temperature of above at least 800 C. To this end, the tubes may be formed of a superalloy. In another approach, the tubes may be formed of nickel-based high-temperature low creep superalloy and chromium.

In another approach, the heat exchanger includes a transport bed heat exchanger for transferring heat between one or more fluid streams as described above and a high heat capacity bulk solid flowing through the heat exchanger 200. The transport bed heat exchanger may include direct heat exchange in which at least one of the pyrolysis stream and the effluent stream directly contacts the bulk solid material. In another approach, the transport bed heat exchanger may include indirect heat exchange in which at least one of a pyrolysis stream and an effluent stream flows through one set of passageways of the heat exchanger 200 in a bulk solid material flows through another set of passageways of the heat exchanger 200 so that heat is transferred from the pyrolysis stream and/or the effluent stream to the bulk solid through components of the heat exchanger.

In another approach, the heat exchanger 200 includes a thermoelectric heat exchanger for converting a portion of the heat from one of the pyrolysis stream and the effluent stream to electricity. The thermoelectric heat exchanger may include a high temperature thermoelectric heat exchanger for operating in the presence of high temperature fluids.

In another approach, the heat exchanger may use a phase transformation fluid capable of transferring energy from the one of the pyrolysis stream and the effluent stream to the phase transformation fluid by transformation of the phase thereof. In one approach, the phase transformation fluid includes a eutectic-eutectic fluid capable of phase transformation upon receiving energy from the pyrolysis stream or the effluent stream. The eutectic-eutectic fluid may operate at or near the eutectic point thereof. Heat transfer may also be achieved via alloy transformations in solid-liquid eutectic mixtures and in eutectic molten salts.

In another approach heat exchanger 200 may provide heat to a circulating heat exchange fluid. The circulating heat exchange fluid may include water, steam, super-heated steam, and a hydrocarbon heat transfer fluid. In one embodiment the hydrocarbon heat transfer fluid is part of a hot oil loop used for carrying heat to other unit operations. Hot oil loops may be used for providing high temperature heat to process users in applications where fired heaters are not appropriate. Conventionally, the so called hot oil is circulated in a loop that includes a fired heater to supply heat to the heat transfer fluid and process users which use heat. In accordance with this approach, the fired heater in the conventional hot oil loop would be replaced by the heat exchanger recovering heat from one of the pyrolysis stream and the effluent stream. The heat exchanger fluid for the hot oil loop may be a synthetic heat transfer fluid which is hydrocarbon fluid that is specially designed to be thermally stable or a non-synthetic heat transfer fluid. Examples of commercially available synthetic heat transfer fluids include, for example, Therminol 66 by Solutia, Dowtherm RP by Dow, and Marlotherm SH by Sasol. Examples of non-synthetic heat transfer fluids include for example diesel fuel or heavy gas oil or any other suitable hydrocarbon stream commonly available and known in the art. The hot oil from heat exchanger 200 may be circulated to process users to provide heat directly or to a boiler to produce steam which in turn may be used to generate electricity, drive compressors, provide heat to process users, or any other use of steam known in the art.

In another approach, the heat exchange fluid may be water and preferably water preheated to its bubble point. In this approach, steam is raised directly from the pyrolysis stream or effluent stream in heat exchanger 200. Advantageously, this directly produces steam, which may be used in other unit operations as described above. In addition, the circulating water rate may be set to minimize the temperature rise of the heat transfer fluid such that saturated steam is produced. In addition, the water may be provided from one or more cooling channels incorporated into reactor 5 for the purpose of cooling the reactor vessel walls. The water may be preheated in the said channels, but not vaporized. The preheated water from the cooling channels may then be directed to heat exchanger 200 to produce steam. The pressure of the circulating water may be increased or reduced as desired to maximize steam production at the desired temperature and pressure.

In one approach, heat exchanger 200 consists of a series of two or more heat exchangers and/or steam drums to produce steam of varying grades. Each heat exchanger in series will produce lower pressure, lower grade steam than the upstream exchangers and steam drums. For example high pressure steam at 600 psig, medium pressure steam at 150 psig, and low pressure steam at 50 psig. The grades of steam may be optimized to produce power, as heat exchange media, or drive other process operations such as a compressor, a turbine, or any combination thereof as desired and understood by one skilled in the art. The steam may be provided in a stream to a power generation device 39 or other process operation. The condensate from the various process users may be recycled back to the cooling channels of reactor 5 or heat exchanger 200. In yet another approach, the heat exchange fluid directed to heat exchanger 200 is steam which is superheated in the subject heat exchanger. The steam may be produced by one of a series of heat exchangers 200 as described above or provided from elsewhere in the process.

In yet another approach, the fluid heated in heat exchanger 200 may be selected from process streams available in the process described with regard to FIG. 1 and FIG. 2. For example one or more of the fuel and oxygen injected into combustion zone 25 via nozzle(s) 30 and feedstock provided to the reactor 5 via feedstock inlet 40 may be preheated in one or more heat exchangers 200 of FIG. 3. Preheating the fuel, oxygen, and feedstock as described above will improve the efficiency of the methane pyrolysis reactor by decreasing the amount of fuel that needs to be burned to achieve a desired level of conversion. In this way heat may be provided directly to the process streams rather than to a utility stream.

A problem that may be encountered when preheating a hydrocarbon containing fuel or feedstock may be thermal decomposition leading to coking of the heat exchanger 200. According to one approach, one method for restricting such coking when preheating the hydrocarbon feedstock includes mixing the feedstock with hydrogen prior to heating. Heating such a mixture may restrict the thermal decomposition of the feedstock.

In one embodiment heat exchanger 200 may be replaced or supplemented by other means of providing heat to the mixture of hydrogen and the hydrocarbon containing stream directed to injectors 30 or 45 such as a fired heater, electric heater, or any other suitable equipment known in the art.

The problem of soot formation is not only a concern in the preheating of hydrocarbon feed prior to injection into the reactor vessel 5. Soot formation is also expected to occur within the reactor vessel; soot is a byproduct of the methane pyrolysis reactions which occur upon injection of the feedstock 40 through injectors 45. The final heating of the hydrocarbon feedstock occurs upon contact with the carrier gas provided by combustion zone 25. As the temperature increases the potential for soot formation is also higher. Injecting the hydrocarbon feed into the methane pyrolysis reactor as a mixed stream containing both the hydrocarbon feedstock and hydrogen is expected to suppress soot formation thereby increasing the yield of the desired products. In a preferred embodiment, a substantially methane feedstock is admixed with a substantially hydrogen stream and heated to a temperature of greater than about 500° C., or greater than about 650° C., or greater than about 800° C. in a preheating zone. The preheating zone may include one or more of a heat exchanger, a fired heater, an electric heater, or any other equipment known in the art for heating a process stream. The preheated mixture is the injected into reactor 5 and converted to among other products acetylene and the acetylene yield may be higher than it otherwise would be if the methane feedstock is not premixed with hydrogen. In addition to the yield benefit of reduced soot and heavy hydrocarbon formation, a lower soot yield is expected to improve the operation of reactor 5 and heating equipment 200 by reducing the deposition of heavy hydrocarbons and fouling materials. Deposition of foulants such as soot may otherwise reduce the efficiency of heat transfer and in the case of reactor 5 potentially lead to hot spots which may weaken the reactor walls or potentially lead to failure. It is anticipated that periodic cleaning will be required to remove soot and other fouling material from the reactors. Therefore, decreasing the rate of soot deposition may improve the on-stream efficiency of the reactor by reducing soot formation, allowing the reactor to be in service for longer between cleanings. Higher on-stream efficiency of the reactor may improve the economic efficiency of the system, providing more time on-stream, translating to a lower operating cost.

Similarly, according to another approach, the oxygen and fuel are pre-mixed upstream of the nozzle(s) 30. In one approach, mixing occurs in channels, which may be maintained at a sufficiently small channel size, so called microchannels, to inhibit the propagation of combustion as disclosed in U.S. Pat. No. 7,192,562, U.S. Pat. No. 7,604,719, U.S. Pat. No. 6,655,829, U.S. Pat. No. 6,863,867, U.S. Pat. No. 7,097,347, and U.S. Pat. No. 7,442,350 incorporated herein in their entirety by reference. In one approach, the microchannels of heat exchanger 200 have an inner diameter of less than about 0.02 cm. These channels may be used to provide a preheated and premixed fuel and oxygen stream to nozzle(s) 30 for combustion in combustion zone 25. Inhibiting combustion by the use of microchannels represents a means of providing a uniformly mixed fuel and oxygen mixture to the combustion chamber at elevated temperature. While the foregoing describes premixing the oxygen and fuel in microchannels, other forms of mixing, with or without the use of microchannels may be made as generally known in the art.

One of the concerns in designing the combustion chamber and the burners or fuel injection nozzles 30 is adequate mixing of the fuel and oxygen and the uniformity of the flow from the combustion zone to the converging-diverging nozzle 50. Mixing the fuel and oxygen in microchannels may advantageously provide a more uniform mixture to the combustion chamber, and thereby provide a more uniform stream to the converging-diverging nozzle 50. A further benefit of incorporating microchannels into the reactor 5 and supplying the fuel and oxygen to the combustion zone 25 is improved control of the position of the flame or combustion reactions. It is desirable to limit combustion to the combustion zone, minimizing oxygen flowing to the downstream zones. Providing a uniform mixture of oxygen and fuel to the combustion zone reduces the likelihood for oxygen to flow directly to nozzle 50 without adequate contact with the fuel, reducing oxidation downstream. Yet another advantage of incorporating microchannels reactor 5 may include improved control of the position of the flame. More particularly, the incorporation of microchannels into the burner design is similar to adding a flame arrester to the burner design, preventing the flame from backing up into the supply lines.

In one example, the reactor effluent stream after pyrolysis in the supersonic reactor 5 has a reduced methane content relative to the methane feed stream ranging from about 15 mol-% to about 95 mol-%. In another example, the concentration of methane ranges from about 40 mol-% to about 90 mol-% and from about 45 mol-% to about 85 mol-% in another example.

In one example the yield of acetylene produced from methane in the feed in the supersonic reactor is between about 40% and about 95%. In another example, the yield of acetylene produced from methane in the feed stream is between about 50% and about 90%. Advantageously, this provides a better yield than the estimated 40% yield achieved from previous, more traditional, pyrolysis approaches.

By one approach, the reactor effluent stream is reacted to form another hydrocarbon compound. In this regard, the reactor effluent portion of the hydrocarbon stream may be passed from the reactor outlet to a downstream hydrocarbon conversion process for further processing of the stream. While it should be understood that the reactor effluent stream may undergo several intermediate process steps, such as, for example, water removal, adsorption, and/or absorption to provide a concentrated acetylene stream, these intermediate steps will not be described in detail herein.

Referring to FIG. 2, the reactor effluent stream having a higher concentration of acetylene may be passed to a downstream hydrocarbon conversion zone 100 where the acetylene may be converted to form another hydrocarbon product. The hydrocarbon conversion zone 100 may include a hydrocarbon conversion reactor 105 for converting the acetylene to another hydrocarbon product. While FIG. 2 illustrates a process flow diagram for converting at least a portion of the acetylene in the effluent stream to ethylene through hydrogenation in hydrogenation reactor 110, it should be understood that the hydrocarbon conversion zone 100 may include a variety of other hydrocarbon conversion processes instead of or in addition to a hydrogenation reactor 110, or a combination of hydrocarbon conversion processes. Similarly, unit operations illustrated in FIG. 2 may be modified or removed and are shown for illustrative purposes and not intended to be limiting of the processes and systems described herein. Specifically, it has been identified that several other hydrocarbon conversion processes, other than those disclosed in previous approaches, may be positioned downstream of the supersonic reactor 5, including processes to convert the acetylene into other hydrocarbons, including, but not limited to: alkenes, alkanes, methane, acrolein, acrylic acid, acrylates, acrylamide, aldehydes, polyacetylides, benzene, toluene, styrene, aniline, cyclohexanone, caprolactam, propylene, butadiene, butyne diol, butandiol, C2-C4 hydrocarbon compounds, ethylene glycol, diesel fuel, diacids, diols, pyrrolidines, and pyrrolidones.

A contaminant removal zone 120 for removing one or more contaminants from the hydrocarbon or process stream may be located at various positions along the hydrocarbon or process stream depending on the impact of the particular contaminant on the product or process and the reason for the contaminants removal, as described further below. For example, particular contaminants have been identified to interfere with the operation of the supersonic flow reactor 5 and/or to foul components in the supersonic flow reactor 5. Thus, according to one approach, a contaminant removal zone is positioned upstream of the supersonic flow reactor in order to remove these contaminants from the methane feed stream prior to introducing the stream into the supersonic reactor. Other contaminants have been identified to interfere with a downstream processing step or hydrocarbon conversion process, in which case the contaminant removal zone may be positioned upstream of the supersonic reactor or between the supersonic reactor and the particular downstream processing step at issue. Still other contaminants have been identified that should be removed to meet particular product specifications. Where it is desired to remove multiple contaminants from the hydrocarbon or process stream, various contaminant removal zones may be positioned at different locations along the hydrocarbon or process stream. In still other approaches, a contaminant removal zone may overlap or be integrated with another process within the system, in which case the contaminant may be removed during another portion of the process, including, but not limited to the supersonic reactor 5 or the downstream hydrocarbon conversion zone 100. This may be accomplished with or without modification to these particular zones, reactors or processes. While the contaminant removal zone 120 illustrated in FIG. 2 is shown positioned downstream of the hydrocarbon conversion reactor 105, it should be understood that the contaminant removal zone 120 in accordance herewith may be positioned upstream of the supersonic flow reactor 5, between the supersonic flow reactor 5 and the hydrocarbon conversion zone 100, or downstream of the hydrocarbon conversion zone 100 as illustrated in FIG. 2 or along other streams within the process stream, such as, for example, a carrier fluid stream, a fuel stream, an oxygen source stream, or any streams used in the systems and the processes described herein.

While there have been illustrated and described particular embodiments and aspects, it will be appreciated that numerous changes and modifications will occur to those skilled in the art, and it is intended in the appended claims to cover all those changes and modifications which fall within the true spirit and scope of the present disclosure and appended claims.

What is claimed is:

1. A method for producing acetylene comprising:
introducing a fuel stream into a combustion zone of a supersonic reactor;
combusting the fuel stream to provide a high temperature carrier stream traveling at a supersonic speed;
introducing a feed stream portion of the hydrocarbon stream into the supersonic reactor; and
premixing at least a portion of the fuel stream with oxygen prior to introduction into the supersonic reactor.

2. The method of claim 1, wherein the fuel and oxygen are premixed in microchannels to restrict combustion of the fuel before it is introduced into the supersonic reactor.

3. The method of claim 2, wherein the microchannels have an inner diameter of less than about 0.02 cm.

4. The method of claim 1, wherein the premixed fuel stream and oxygen are introduced into the supersonic reactor via nozzles.

5. The method of claim 1, wherein at least one of the portion of the fuel stream and a portion of the oxygen are preheated prior to introduction into the supersonic reactor.
